(19) United States
(12) Patent Application Publication
(10) Pub. No.: US 2015/0071964 A1
(43) Pub. Date: Mar. 12, 2015

(54) METHODS AND COMPOSITIONS FOR VIRAL VECTORED VACCINES

(71) Applicant: De-Chu Tang, Birmingham, AL (US)

(72) Inventor: De-Chu Tang, Birmingham, AL (US)

(73) Assignee: VAXIN INC., Gaithersburg, MD (US)

(21) Appl. No.: 14/478,040

(22) Filed: Sep. 5, 2014

Related U.S. Application Data
(60) Provisional application No. 61/874,505, filed on Sep. 6, 2013.

(51) Int. Cl. C12N 15/86

(52) U.S. Cl. CPC C12N 15/86; C12N 2710/10034; C12N 2710/10042
USPC 424/199.1

(57) ABSTRACT

Methods and compositions are provided herein for non-invasive administration of an adenoviral vector (Ad-vector) vaccine with an adjuvant, such as a TLR3 agonist. These methods provide, for example, an increase in the immune response to the vaccine, an increase in the immunogenicity of the Ad-vector vaccine, an antigen sparing effect and improved safety with an effective protective immune response to the vaccine.

### RELATED APPLICATIONS AND INCORPORATION BY REFERENCE

This application claims benefit of and priority to U.S. provisional patent application Ser. No. 61/874,505 filed Sep. 6, 2013.

All documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The disclosure relates to methods and compositions for non-invasive administration of an adenovirus vector containing and expressing a heterologous gene, and administration of an adjuvant.

### BACKGROUND

There are several noteworthy reasons for utilizing recombinant Ad vector as a vaccine carrier. These include 1) Ad vectors are capable of transducing both mitotic and postmitotic cells in situ (Shi 1999); 2) stocks containing high titers of virus (greater than 10¹² pfu (plaque forming unit) per ml)) can be prepared, making it possible to transduce cells in situ at high multiplicity of infection (MOI); 3) the vector is safe based on its long-term use as a vaccine; 4) the virus is capable of inducing high levels of transgene expression (at least as an initial burst) and 5) the vector can be engineered to a great extent with versatility. Recombinant Ad vectors have been utilized as vaccine carriers by intranasal, epicutaneous, intratracheal, intraperitoneal, intravenous, subcutaneous and intramuscular routes.

Ad-vectored nasal vaccine appears to be more effective in eliciting an immune response than injection of DNA or topical application of Ad (Shi et al. (2001) J. Virol. 75:11474-11482). Previously reported results have shown that the potency of the E1/E3 defective Ad5 vector as a nasal vaccine carrier is not suppressed by an preexisting immunity to Ad (Xiang et al. (1996) Virology 219(1) 220-7; Shi et al. 2001).

Ad-based vaccines mimic the effects of natural infections in their ability to induce major histocompatibility complex (MHC) class I restricted T-cell responses, yet eliminate the possibility of reversion back to virulence because only a subfragment of the pathogen's genome is expressed from the vector. This “selective expression” may solve the problem of differentiating vaccinated-but-uninfected animals from their infected counterparts, because the specific markers of the pathogen not encoded by the vector can be used to discriminate the two events. Notably, propagation of the pathogen is not required for generating vectored vaccines because the relevant antigen genes can be amplified and cloned directly from field samples (Rajakumar et al., 1990). This is particularly important for production of highly virulent AI strains, such as H5N1, because this strain is too dangerous and difficult to propagate (Wood et al., 2002).

U.S. Pat. No. 4,349,538 (Hilton B LEVY) and U.S. Pat. No. 7,439,349 (Andres M. Salazar) relates to the preparation and clinical use of Poly-ICLC. Polyinosinic-Polycytidylic acid stabilized with polylysine and carboxymethylcellulose (Poly-ICLC) is a synthetic complex of polyinosinic and polycytidylic acid (double-stranded RNA (dsRNA)), stabilized with polylysine and carboxymethyl cellulose that was used as an interferon inducer at high doses (up to 300 mcg/kg IV) in short-term cancer trials some years ago. This gave mixed results with moderate toxicity, and the use of Poly-ICLC was generally abandoned when recombinant interferons became available. However, lower dose (10 to 50 mcg/kg) poly-ICLC results in a broader host defense stimulation, and enhanced clinical activity with little or no toxicity. As such it represents an example of broad spectrum host-targeted therapeutics, in contrast to conventional antibiotics, antiviral or antineoplastic agents that target specific organisms or tumors. (Salazar, Levy et al. 1996) (Ewel, Urba et al. 1992) (Levy and Salazar 1992) (Talmadge and Hartman 1985) (Maluish, Reid et al. 1985).

There are at least five interrelated biological actions of poly-ICLC (HILTONOL®), any of which (alone or in combination) might be responsible for its antiviral activity. These are 1) its induction of interferons; 2) its broad immune enhancing effect; 3) its activation of specific enzymes, especially oligoadenylate synthetase (OAS) and the p68 protein kinase (PKR); 4) its broad gene regulatory actions and 5) its activation of one or more toll-like receptors (TLRs) including TLR3 (Proc Natl Acad Sci USA. 2008 Feb. 19; 105(7): 2574-2579).

Poly-ICLC also has a vaccine-boosting or adjuvant effect, with increased antibody and cellular immune response to antigen. For example, administration of low doses of Poly-ICLC along with swine flu vaccination in monkeys dramatically accelerates and increases antibody production. The complex interactions of the dsRNAs and the interferons in this regard are still incompletely understood, yet this seemingly paradoxical dual role of Poly-ICLC as an antiviral agent and immune enhancer is consistent with its function in establishing an immediate defense system against viral attack while at the same time stimulating the establishment of long term immunity.

However, there still remains a need for non-invasive administration of an adenovirus vectored vaccine and adjuvant that will increase the immunogenicity of the vaccine and provide protection against an infectious antigen challenge. An additional advantage of the adjuvant could be in its antigen sparing activity, i.e., the ability to achieve protective vaccine titers at a lower vaccine dose than that achievable using the vaccine alone.

Citation or identification of any document in this application is not an admission that such document is available as prior art to the present invention.

### SUMMARY

In certain embodiments are provided methods for increasing immunogenicity of an adenoviral vector (Ad-vector) vaccine in an animal, wherein the method may comprise administering the Ad-vector in a non-invasive mode to the animal, wherein the vaccine may comprise and expresses a gene of interest; and, administering an Ad-vector vaccine adjuvant in a non-invasive mode to the animal at the same time (co-administration) or within 24 hours of administering the Ad-vector vaccine, wherein the Ad-vector vaccine adjuvant is poly-ICLC or a TLR3 agonist, wherein administration of the poly-ICLC or a TLR3 agonist increases the immunogenicity of the Ad-vector vaccine as compared to the Ad-vector vaccine administered without the poly-ICLC or a TLR3 agonist.

In further embodiments are provided methods for inducing a protective immune response in an animal in need thereof, wherein the method may comprise administering the adenoviral vector (Ad-vector) in a non-invasive mode to the animal, wherein the vaccine may comprise and expresses an antigen of interest; and, administering an Ad-vector vaccine adjuvant in a non-invasive mode to the animal at the same time (co-administration) or within 24 hours of administering the Ad-vector vaccine, wherein the Ad-vector vaccine adjuvant is poly-ICLC or a TLR3 agonist, wherein induction of the immune response provides protection against challenge from infection of the antigen.

In other embodiments are provided methods for increasing the immune response rate to an adenoviral vector (Ad-vector) vaccine in an animal, wherein the method may comprise administering the Ad-vector vaccine in a non-invasive mode to the animal, wherein the vaccine may comprise and expresses an antigen of interest; and, administering an Ad-vector vaccine adjuvant in a non-invasive mode to the animal at the same time (co-administration) or within 24 hours of administering the Ad-vector vaccine, wherein the Ad-vector vaccine adjuvant is poly-ICLC or a TLR3 agonist, wherein administration of the poly-ICLC or a TLR3 agonist increases the immune response rate to the Ad-vector vaccine as compared to an Ad-vectored vaccine administered without the poly-ICLC or a TLR3 agonist.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The accompanying drawings, which are incorporated into and constitute a part of this specification, illustrate one or more embodiments of the present disclosure and, together with the detailed description and examples sections, serve to explain the principles and implementations of the disclosure.

FIG. 1 shows Kaplan-Meier survival curves for groups of mice vaccinated on a single occasion, 28 days before challenge infection. The 10⁸ dose of Ad-VN.H5 vaccine provided 100% protection from challenge infection, regardless of the concentration (5, 15, or 50 μg in a 10 μl volume) of poly ICLC (FIG. 1A). In addition, all four doses of Ad-VN.H5 (1.2×10⁶, 1.2×10⁷, 1.2×10⁸, or 3.5×10⁸ ifu/50 μl) combined with 15 μg of poly-ICLC provided 100% protection from challenge infection (FIG. 1B). The group receiving the AdE also showed significant protection, although some mortality was observed (FIGS. 1A & 1B).

FIG. 2 shows mean body weight changes for groups of mice vaccinated 28 days before challenge infection. All mice receiving the 10⁸ AdVN.H5 vaccines were protected from significant weight loss. However, the group receiving the lowest dose (5 μg in 10 μl volume) of poly-ICLC showed the best protection (FIG. 2A). Mice vaccinated with the 10⁶ dose of AdVN.H5 combined with the 15 μg dose of poly-ICLC also showed significant protection from weight loss. In addition, groups receiving the 10⁷ and 10⁸ doses of vaccine showed significant differences in mean body weights compared to placebo (FIG. 2B).

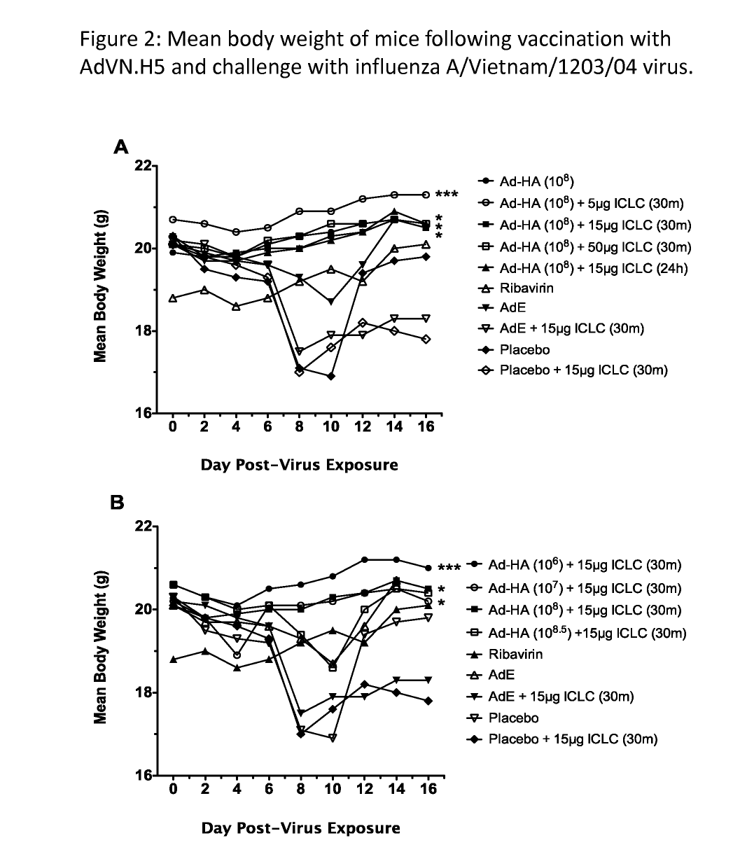

FIG. 3 shows results for hemagglutination inhibition (HAI) assays on serum at day 14 following vaccination. On day 14 post-vaccination, the groups receiving the non-adjuvanted 10⁸ dose of AdVN.H5 and the 10⁸ AdVN.H5 dose combined with poly-ICLC at 24 hours post-vaccination showed significant increases over placebo (FIG. 3A). Results of the vaccine dose titration indicate that only the group receiving the highest (10^{8.5}) dose of AdVN.H5 showed a significant difference compared to placebo (FIG. 3B).

FIG. 4 shows results for hemagglutination inhibition (HAI) assays on serum at day 28 following vaccination. On day 28 post-vaccination, all groups receiving the 10⁸ dose of AdVN.H5 showed significant increases over placebo (FIG. 4A). However, in the dose titration, only groups receiving the two highest doses of AdVN.H5 showed significant increases in HAI titer (FIG. 4B).

FIG. 5 shows the levels of sIgA in lung lavage on day 14 following vaccination. On day 14 post-vaccination, all groups receiving 10⁸ AdVN.H5 showed significant increases over placebo, except for the group receiving AdVN.H5 containing 50 μg of poly-ICLC (FIG. 5A). Only groups receiving the 10⁸ and 10^{8.5} doses of vaccine combined with the 15 μg dose of poly-ICLC showed significant increases in IgA (FIG. 5B). In addition, the level of IgA induced by the 10^{8.5} AdVN.H5 vaccine, when combined with 15 μg of poly-ICLC was significantly higher than all other vaccine formulations on day 14 post-vaccination.

FIG. 6 shows the levels of sIgA in lung lavage on day 28 following vaccination. On day 28 post-vaccination, all groups receiving the 10⁸ dose of AdVN.H5 showed significant increases over placebo (FIG. 6A). However, only groups receiving the 10⁸ and 10^{8.5} AdVN.H5 vaccines combined with the 15 μg dose of poly-ICLC showed significant increases (FIG. 6B). In addition, the level of IgA induced by the 10⁸ AdVN.H5 vaccine combined with 15 μg of poly-ICLC administered 24 h post-vaccination was significantly higher than all other vaccine formulations on day 28 post-vaccination.

FIG. 7 shows the number of IFN-γ producing cells isolated and cultured from lung lavage on day 28 following vaccination. On day 14 post-vaccination, only the group receiving the 10⁸ dose of AdVN.H5 combined with poly-ICLC at 24 hours post-vaccination showed a significant increase in the number of IFN-γ producing cells (FIG. 7A). No significant differences in IFN-γ producing cells were observed for groups treated with different doses of AdVN.H5 (FIG. 7B).

FIG. 8 shows the number of IFN-γ producing cells isolated and cultured from lung lavage on day 28 following vaccination. On day 28 post-vaccination, all groups vaccinated with 10⁸ AdVN.H5 combined with poly-ICLC showed significant increases in IFN-γ producing cells (FIG. 8A). However, only the groups receiving the 10⁸ and 10^{8.5} doses of AdVN.H5 vaccine combined with the 15 μg dose of poly-ICLC showed significant increases (FIG. 8B).

FIG. 9 shows the number of IL-4 producing cells isolated and cultured from lung lavage on day 14 following vaccination. On day 14 post-vaccination, only the group receiving 10⁸ AdVN.H5 combined with the 15 μg dose of poly-ICLC showed a significant increase in the number of IL-4 producing cells.

FIG. 10 shows the number of IL-4 producing cells isolated and cultured from lung lavage on day 28 following vaccination. On day 28 post-vaccination, an increase in the number of IL-4 producing cells was observed in all groups. Therefore, no significant differences were observed among vaccine groups.

### DETAILED DESCRIPTION OF THE INVENTION

The present invention provides methods and compositions for inducing a protective immune response against pathogens in a subject in need thereof following non-invasive administration of an adenoviral vectored (Ad-vectored) vaccine and a double stranded (ds) RNA polynucleotide or TLR 3 agonist as an adjuvant (herein referred to as “Ad-vector vaccine adjuvant”). Surprisingly, using a synthetic dsRNA polynucleotide, such as poly ICLC (HILTONOL®), with known antiviral activity, as an adjuvant administered by the same route as the vaccine and at the same time (co-administration) or within 24 hours (0-24 hrs) of the vaccine, significantly increased the immunogenicity of the Ad-vectored vaccine. This improvement in the immunogenicity of the vaccine results in an improvement in survival rate following challenge infection by a live virus, such as H5N1 influenza virus.

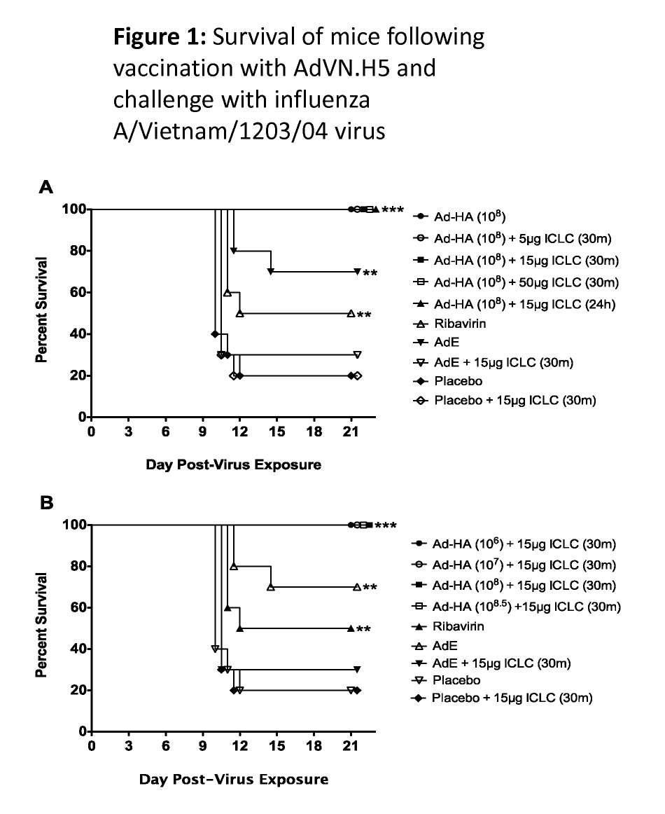

In certain embodiments, 100% protection against a challenge infection was observed, See FIG. 1B. Ad-vectored vaccines at the 10⁶ dose, as used herein, are not typically protective following challenge infection with live virus when used without the present adjuvant. However, co-administration (0-24 hours) of the Ad-vectored vaccine adjuvant results in at least 90% protection, and in certain embodiments provides 100% protection from an infectious challenge.

As used herein an Ad-vectored vaccine may comprise and expresses a gene of interest such as an influenza or anthrax antigen or fragment thereof. The instant disclosure provides a significant improvement in the effectiveness, including lowering the dose needed, of using an Ad-vectored vaccine for providing protection against pathogens wherein the vaccine is administered non-invasively.

Accordingly, in one embodiment provided herein are methods for increasing the immunogenicity of an Ad-vectored vaccine when administered non-invasively and the Ad-vectored vaccine adjuvant is administered at the same time (co-administration) or within 24 hrs, wherein administration of the poly-ICLC or a TLR3 agonist (as adjuvant) increases the immunogenicity of the Ad-vector vaccine as compared to the Ad-vector vaccine administered without the poly-ICLC or a TLR3 agonist.

In another embodiment provided herein are methods for inducing a protective immune response in a subject in need thereof, wherein the Ad-vectored vaccine is administered non-invasively and the Ad-vectored vaccine adjuvant is administered at the same time (co-administration) or within 24 hrs of the vaccine administration, wherein induction of the immune response provides protection against infectious challenge of the antigen.

In yet another embodiment provided herein are methods for increasing immune response rate to an adenoviral vector (Ad-vector) vaccine in an animal, wherein the Ad-vectored vaccine is administered non-invasively and the Ad-vectored vaccine adjuvant is administered at the same time (co-administration) within 24 hrs of the vaccine administration, wherein administration of the poly-ICLC or a TLR3 agonist (as adjuvant) increases the immune response rate to the Ad-vector vaccine as compared to an Ad-vectored vaccine administered without the poly-ICLC or a TLR3 agonist. As used herein, rate refers to the time between administering the vaccine and eliciting an immune response; the shorter the time the faster the rate.

Example 1 provides the use of a synthetic dsRNA poly-ICLC (Hiltonol) as adjuvant to increase the immunogenicity of an Ad5-vectored influenza virus HA vaccine (Ads-VN1203/04.H5) against challenge infection with highly pathogenic A/Vietnam/1203/04 (H5N1) avian influenza virus in mice. In a comparison of AdVN.H5 vaccines administered 30 min or 24 hours prior to administration of different doses of poly-ICLC, all treatment groups receiving the 10⁸ dose of Ad-VN.H5 provided 100% protection from challenge infection, regardless of the concentration of poly-ICLC. In addition, all four doses of AdVN.H5 (1.2×10⁶, 1.2×10⁷, 1.2×10⁸, or 3.5×10⁸ ifu/50 μl) vaccine administered 30 min prior to administration of 15 μg poly-ICLC provided 100% protection from challenge infection. The AdE (no influenza antigen) also showed significant protection, although some mortality was observed. The protection afforded by the empty AdE vector was surprising, and suggests more than one mechanism of action for this specific Ad5 vector.

All treatment groups receiving the 10⁸ dose of Ad-VN.H5 protected mice from significant weight loss, regardless of the concentration of poly-ICLC. However, the 5 μg dose of poly-ICLC showed the best protection. Upon comparing four doses of AdVN.H5 vaccines, the 10⁶ dose of AdVN.H5 combined with the 15 μg dose of poly-ICLC showed the best protection from weight loss. Therefore, the survival and weight loss data indicate that the 10⁸ dose of AdVN.H5 is protective regardless of the concentration of poly-ICLC. However, even lower doses of vaccine may be protective if combined with adjuvant.

As used herein, the terms “a” or “an” are used, as is common in patent documents, to include one or more than one, independent of any other instances or usages of “at least one” or “one or more.”

As used herein, the term “or” is used to refer to a nonexclusive or, such that “A or B” includes “A but not B,” “B but not A,” and “A and B,” unless otherwise indicated.

As used herein, the term “about” is used to refer to an amount that is approximately, nearly, almost, or in the vicinity of being equal to or is equal to a stated amount, e.g., the state amount plus/minus about 5%, about 4%, about 3%, about 2% or about 1%.

As used herein, the term, “adjuvant” refers to a pharamacological or immunological agent that modifies the effect of another agent, such as enhancing the immune response to a supplied antigen from a vaccine.

The terms “Ad-vector vaccine” or “Ad-vectored vaccine” as used herein interchangeably, refer to an adenoviral vector which may comprise a gene of interest which encodes an antigen. The adenovirus may be any adenovirus, such as but not limited to, a human adenovirus, a bovine adenovirus, a canine adenovirus, a non-human primate adenovirus, a chicken adenovirus, or a porcine or swine adenovirus.

As used herein, the term, “Ad-vector vaccine adjuvant” refers to a double stranded (ds) RNA polynucleotide or TLR 3 agonist that when co-administered, or administered within 24 hours of the ad-vectored vaccine acts as an adjuvant for enhancing the immune response of the Ad-vector vaccine. In certain embodiments the Ad-vector vaccine adjuvant is poly-IC (poly inosinic-polycytidilic acid), poly-ICLC, poly-IC(12)U or poly-IC(12)G. In other embodiments, the Ad-vector vaccine adjuvant are dsRNA molecules with base modifications or modifications to the nucleic acid backbone, sugar moiety, or other sites in one or both strands of the nucleic acids, or which are incorporated in liposomes or polymers, and which bind to and/or activate immune cells through an interaction with the double stranded RNA pattern recognition receptors (PRR), including but not limited to Toll-Like Receptor 3 (TLR3).

As used herein, the term “human adenovirus” is intended to encompass all human adenoviruses of the Adenoviridae family, which include members of the Mastadenovirus genera. To date, over fifty-one human serotypes of adenoviruses have been identified (see, e.g., Fields et al., Virology 2, Ch. 67 (3d ed., Lippincott-Raven Publishers)). The adenovirus may be of serogroup A, B, C, D, E, or F. The human adenovirus may be a serotype 1 (Ad 1), serotype 2 (Ad2), serotype 3 (Ad3), serotype 4 (Ad4), serotype 5 (Ad5), serotype 6 (Ad6), serotype 7 (Ad7), serotype 8 (Ad8), serotype 9 (Ad9), serotype 10 (Ad10), serotype 11 (Ad11), serotype 12 (Ad12), serotype 13 (Ad13), serotype 14 (Ad14), serotype 15 (Ad15), serotype 16 (Ad16), serotype 17 (Ad17), serotype 18 (Ad18), serotype 19 (Ad19), serotype 19a (Ad19a), serotype 19p (Ad19p), serotype 20 (Ad20), serotype 21 (Ad21), serotype 22 (Ad22), serotype 23 (Ad23), serotype 24 (Ad24), serotype 25 (Ad25), serotype 26 (Ad26), serotype 27 (Ad27), serotype 28 (Ad28), serotype 29 (Ad29), serotype 30 (Ad30), serotype 31 (Ad31), serotype 32 (Ad32), serotype 33 (Ad33), serotype 34 (Ad34), serotype 35 (Ad35), serotype 36 (Ad36), serotype 37 (Ad37), serotype 38 (Ad38), serotype 39 (Ad39), serotype 40 (Ad40), serotype 41 (Ad41), serotype 42 (Ad42), serotype 43 (Ad43), serotype 44 (Ad44), serotype 45 (Ad45), serotype 46 (Ad46), serotype 47 (Ad47), serotype 48 (Ad48), serotype 49 (Ad49), serotype 50 (Ad50), serotype 51 (Ad51), or combinations thereof, but are not limited to these examples. In certain embodiments, the adenovirus is serotype 5 (Ad5).

As used herein, the term “non-invasive administration” refers to administration of the Ad-vector vaccine via topical application and/or via mucosal and/or via skin and/or via intranasal administration.

As used herein, the term “TLR 3 agonist” refers to a synthetic toll-like receptor 3 (TLR 3) ligand which activates the TRIF dependent signaling pathway in dendritic cells and B cells. TLR3 recognizes double-stranded RNA (dsRNA) of viruses and its synthetic analog Polyinosine-polycytidylic acid (poly(I:C)). TLR 3 agonists include, but are not limited to, poly-IC, poly-ICLC, poly-IC(12)U and poly-AU.

The present disclosure is directed to a method of non-invasive genetic immunization or treatment in an animal, which may comprise the step of: contacting the animal in a non-invasive mode (e.g., skin/mucosal/intranasal area of the animal) with an Ad-vector vaccine and an Ad-vector vaccine adjuvant (Poly-ICLC and/or a TLR 3 agonist) wherein the amount of the vaccine and the adjuvant together is an amount effective to induce a protective immune response in the animal.

In certain embodiments the dosage of the Ad-vector vaccine to induce a protective immune response is lower than compared to an Ad-vectored vaccine used without the present Ad-vectored vaccine adjuvant. Dosage of the Ad-vector vaccine when used with Poly-ICLC or a TLR3 agonist may range from about 10⁶ to about 10¹² ifu or pfu. In one aspect the dose of Ad-vector vaccine administered to the animal is about, or at least about, 10⁶ ifu or pfu. In another aspect the dose of Ad-vector vaccine administered to the animal is about, or at least about, 10⁷ ifu or pfu. In yet another aspect, the dose of Ad-vector vaccine administered to the animal is about, or at least about, 10⁸ ifu or pfu. In another aspect the dose of Ad-vector vaccine administered to the animal is about, or at least about, 10⁹ ifu or pfu. In another aspect the dose of Ad-vector vaccine administered to the animal is about, or at least about, 10¹⁰ ifu or pfu. In yet another aspect, the dose of Ad-vector vaccine administered to the animal is about, or at least about, 10¹¹ ifu or pfu. In another aspect the dose of Ad-vector vaccine administered to the animal is about, or at least about, 10¹² ifu or pfu.

One of skill in the art understands that an effective dose in a mouse (or any animal used in pre-clinical studies) may be scaled for larger animals such as humans. In this way, through allometric scaling (also referred to as biological scaling) a dose in a human may be extrapolated from a dose in a pre-clinical animal to obtain an equivalent dose based on body weight or body surface area of the animal. A dose of the Ad-vector vaccine in a human may be about 10⁹ to about 10¹² ifu or pfu. In one aspect the dose of Ad-vector vaccine administered to the human is about, or at least about, 10⁹ ifu or pfu. In one aspect the dose of Ad-vector vaccine administered to the human is about, or at least about, 10¹⁰ ifu or pfu. In another aspect the dose of Ad-vector vaccine administered to the human is about, or at least about, 10¹¹ ifu or pfu. In yet another aspect, the dose of Ad-vector vaccine administered to the human is about, or at least about, 10¹² ifu or pfu.

In certain embodiments, the immunogenicity of the Ad-vector vaccine is increased as compared to the Ad-vector vaccine used without the Ad-vectored vaccine adjuvant. Protective immunogenicity may be measured, for example, by comparing titer of neutralizing antibody, wherein an increase in titer of neutralizing antibody represents an increase in immunogenicity of the vaccine. Increased immunogenicity may also be measured by protection, or survival rate, following antigen challenge. In one aspect, the combination of Ad-vector vaccine and adjuvant provides at least about 90% protection from challenge. In another aspect, the combination of Ad-vector vaccine and adjuvant provides at least about 95% protection from challenge. In certain embodiments, the combination of Ad-vector vaccine and adjuvant provides about 100% protection from challenge.

In certain other embodiments, the safety of the Ad-vector vaccine is improved as compared to the Ad-vector vaccine used without the Ad-vectored vaccine adjuvant. Improvement in safety of the vaccine may be measured, for example, by weight loss, wherein an improvement in weight loss (less weight is lost following administration of the vaccine and adjuvant) represents an improvement in safety of the vaccine.

In certain other embodiments, the mucosal immunity in response to administration of the Ad-vector vaccine is increased as compared to the Ad-vectored vaccine used without the Ad-vectored vaccine adjuvant. Mucosal immunity may be measured, for example, by comparing titer of secretory IgA, wherein an increase in sIgA represents an increase in mucosal immunity.

In certain other embodiments, the immune response rate following administration of the Ad-vector vaccine is increased as compared to the Ad-vectored vaccine used without the present Ad-vectored vaccine adjuvant. Immune response time following administration of the Ad-vectored vaccine may be measured, for example, by comparing IFNgamma secreting cell numbers across days post vaccination, wherein an increase in immune cells at an earlier time point represents an increase in the immune response rate to administration of the Ad-vector vaccine.

The Ad-vector vaccine adjuvant (Poly-ICLC or a TLR3 agonist) may be co-administered (time 0) with the Ad-vector vaccine, or shortly thereafter as is feasible, or at any time within, and including, 24 hours post Ad-vector vaccine administration. In certain embodiments the Ad-vector vaccine adjuvant is administered, 5 minutes, 10 minutes, 15 minutes, 20 minutes, 25 minutes, 30 minutes, 45 minutes, 1 hour, 90 minutes, 2 hours, 2.5 hours, 3 hours, 3.5 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or any combination thereof, following administration of the Ad-vector vaccine. The adjuvant dose may be administered once, or multiple times during the 24 hours following administration of the Ad-vector vaccine. In certain embodiments, the Ad-vector vaccine is co-administered with the Ad-vector vaccine adjuvant. In one aspect the Ad-vector vaccine is co-administered with poly-ICLC, poly-IC(12)U or a TLR3 agonist as the Ad-vector vaccine adjuvant.

The adjuvant dose is an amount, that when administered with or following the Ad-vector vaccine, induces an enhanced protective immune response as compared to administration of the Ad-vector vaccine without the adjuvant. In certain embodiments, the dose of the adjuvant includes, but is not limited to, about 5 ug to about 50 ug. In one aspect, the dose of the adjuvant is about 5 ug to about 25 ug. In another aspect, the dose of the adjuvant is about 5 ug to about 15 ug. In one aspect, the dose of the adjuvant is a low dose of about 5 ug. In another aspect, the dose of the adjuvant is a high dose of about 25 to about 50 ug. In yet another aspect, the dose of the adjuvant is a medium dose of about 15 ug. The dose may be represented as a final dose administered to the animal, by weight of the animal or by surface area of the animal.

One of skill in art understands that a dose used for a pre-clinical animal may be scaled for larger animals, such as humans, by allometric scaling based on body weight or body surface area. In this instant, a dose of adjuvant for a human may be about 1 mg to about 5 mg. In one aspect the amount of adjuvant administered to a human may be about 1 mg, about 1.5 mg, about 2 mg, about 2.5 mg, about 3 mg, about 3.5 mg, about 4 mg, about 4.5 mg or about 5 mg. An appropriate dose for each patient may be accurately calculated and administered with the Ad-vector vaccine.

Any adenoviral vector (Ad-vector) known to one of skill in art, and prepared for non-invasive application, which may comprise and express an immunogenic antigen may be used with the methods of this disclosure. Such Ad-vectors include any of those in U.S. Pat. Nos. 6,706,693; 6,716,823; 6,348,450; or US Patent Publ. Nos. 2003/0045492; 2004/0009936; 2005/0271689; 2007/0178115; 2012/0276138 (herein incorporated by reference in entirety).

In certain embodiments the recombinant adenovirus vector is non-replicating. In certain embodiments the recombinant adenovirus vector may include E1-defective, E3-defective, and/or E4-defective adenovirus vectors, or the “gutless” adenovirus vector in which all viral genes are deleted. The E1 mutation raises the safety margin of the vector because E1-defective adenovirus mutants are replication incompetent in non-permissive cells. The E3 mutation enhances the immunogenicity of the antigen by disrupting the mechanism whereby adenovirus down-regulates MHC class I molecules. The E4 mutation reduces the immunogenicity of the adenovirus vector by suppressing the late gene expression, thus may allow repeated re-vaccination utilizing the same vector. The “gutless” adenovirus vector replication requires a helper virus and a special human 293 cell line expressing both E1a and Cre, a condition that does not exist in natural environment; the vector is deprived of all viral genes, thus the vector as a vaccine carrier is non-immunogenic and may be inoculated for multiple times for re-vaccination. The “gutless” adenovirus vector also contains 36 kb space for accommodating transgenes, thus allowing co-delivery of a large number of antigen genes into cells. Specific sequence motifs such as the RGD motif may be inserted into the H-I loop of an adenovirus vector to enhance its infectivity. An adenovirus recombinant may be constructed by cloning specific transgenes or fragments of transgenes into any of the adenovirus vectors such as those described below. The adenovirus recombinant vector is used to transduce epidermal cells of a vertebrate in a non-invasive mode for use as an immunizing agent.

In other embodiments, combinations of adenovirus vectors are provided. For example, an empty Ad-vector (E1/E3 deleted with no insert) may be sequentially or simultaneously administered to a patient in need thereof along with another vector, such as an Ad-vector, which may be E1/E3 deleted with an insert, such as an exogenous and/or heterologous gene as herein described. Without being bound by theory, the empty Ad-vector (E1/E3 deleted with no insert) may initially elicit a rapid immune response wherein a vector expressing an exogenous and/or heterologous gene, such as an antigen or epitope, may elicit an additional protective response.

In certain embodiments, non-invasive administration of the Ad-vector includes, but is not limited to, topical application to the skin, and/or intranasal and/or mucosal and/or perlingual and/or buccal and/or oral and/or oral cavity administration. Dosage forms for the application of the Ad-vector vaccine may include liquids, ointments, powders and sprays. The active component may be admixed under sterile conditions with a physiologically acceptable carrier and any preservative, buffers, propellants, or absorption enhancers as may be needed.

If nasal or respiratory (mucosal) administration is desired, compositions may be in a form and dispensed by a squeeze spray dispenser, pump dispenser, multi-dose dispenser, dropper-type dispenser or aerosol dispenser. Such dispensers may also be employed to deliver the composition to oral or oral cavity (e.g., buccal or perlingual) mucosa. Aerosols are usually under pressure by means of a hydrocarbon. Pump dispensers may preferably dispense a metered dose or, a dose having a particular particle size.

While non-invasive delivery is desirable in all instances of administration including the adjuvant, the methods may be used in conjunction with invasive deliveries; and, such methods may generally be used as part of a prime-boost regimen. For instance, the methods may be used as part of a prime-boost regimen wherein the non-invasive inventive method is administered prior to or after or concurrently with another administration such as another non-invasive or an invasive administration of the same or a different immunological or therapeutic ingredient, e.g., before, during or after the non-invasive administration, there is administration by injection of a different vaccine or immunological composition for the same or similar pathogen such as a whole or subunit vaccine or immunological composition for the same or similar pathogen whose antigen or epitope of interest is expressed by the vector in the non-invasive administration.

An immunological effective amount, as used herein refers to an amount or concentration of the Ad-vector encoding the gene of interest, that when administered to a subject, produces an immune response to the gene product of interest (Ad-vector vaccine). The Ad-vector vaccines of the present disclosure may be administered to an animal either alone or as part of an immunological composition.

The immunogenic compositions may contain pharmaceutically acceptable flavors and/or colors for rendering them more appealing, especially if they are administered orally (or buccally or perlingually); and, such compositions may be in the form of tablets or capsules that dissolve in the mouth or which are bitten to release a liquid for absorption buccally or perlingually (akin to oral, perlingual or buccal medicaments for angina such as nitroglycerin or nifedimen). The viscous compositions may be in the form of gels, lotions, ointments, creams and the like (e.g., for topical and/or mucosal and/or nasal and/or oral and/or oral cavity and/or perlingual and/or buccal administration), and will typically contain a sufficient amount of a thickening agent so that the viscosity is from about 2500 to 6500 cps, although more viscous compositions, even up to 10,000 cps may be employed.

Liquid preparations are normally easier to prepare than gels, other viscous compositions, and solid compositions. Additionally, liquid compositions are somewhat more convenient to administer, especially by orally or buccally or perlingually, to animals, children, particularly small children, and others who may have difficulty swallowing a pill, tablet, capsule or the like, or in multi-dose situations. Viscous compositions, on the other hand, may be formulated within the appropriate viscosity range to provide longer contact periods with mucosa, such as the lining of the stomach or nasal mucosa or for perlingual or buccal or oral cavity absorption.

The Ad-vector may be matched to the host or may be a vector that is interesting to employ with respect to the host or animal because the vector may express both heterologous or exogenous and homologous gene products of interest in the animal; for instance, in veterinary applications, it may be useful to use a vector pertinent to the animal, for example, in canines one may use canine adenovirus; or more generally, the vector may be an attenuated or inactivated natural pathogen of the host or animal upon which the method is being performed. One skilled in the art, with the information in this disclosure and the knowledge in the art, may match a vector to a host or animal without undue experimentation.

Therefore, in additional to human vaccines described herein, the method of the disclosure may be used to immunize animal stocks. The term animal means all animals including humans. Examples of animals include humans, cows, dogs, cats, goats, sheep, birds and pigs, etc. Since the immune systems of all vertebrates operate similarly, the applications described may be implemented in all vertebrate systems.

In certain embodiments, the animal is a vertebrate such as a mammal, bird, reptile, amphibian or fish; a human, or a companion or domesticated or food-producing or feed-producing or livestock or game or racing or sport animal such as a cow, a dog, a cat, a goat, a sheep or a pig or a horse, or fowl such as turkey, ducks or chicken. In a specific embodiment the vertebrate is a human. In another specific embodiments, the vertebrate is a bird.

In certain embodiments, the Ad-vector expresses a gene encoding an influenza antigen, a respiratory syncytial virus (RSV) antigen, a HIV antigen, a SIV antigen, a HPV antigen, a HCV antigen, a HBV antigen, a CMV antigen or a Staphylococcus antigen. The influenza may be swine influenza, seasonal influenza, avian influenza, H1N1 influenza or H5N1 influenza.

In other embodiments, the Ad-vector expresses a gene which encodes influenza hemagglutinin, influenza nuclear protein, influenza M2, influenza neuraminidase, tetanus toxin C-fragment, anthrax protective antigen, anthrax lethal factor, rabies glycoprotein, HBV surface antigen, HIV gp 120, HW gp 160, malaria CSP, malaria SSP, malaria MSP, malaria pfg, mycobacterium tuberculosis HSP or a mutant thereof.

In certain embodiments the protective immune response in the animal is induced by genetic vectors expressing genes encoding antigens of interest in the animal's cells. In certain other embodiments, the animal's cells are epidermal cells. In another embodiment, the Ad-vector is used as a prophylactic vaccine or a therapeutic vaccine. In another embodiment, the genetic vector may comprise genetic vectors capable of expressing an antigen of interest in the animal's cells.

In certain embodiments, the Ad-vector further may comprise a gene selected from the group consisting of co-stimulatory genes and cytokine genes. In this instant the gene is selected from the group consisting of a GM-CSF gene, a B7-1 gene, a B7-2 gene, an interleukin-2 gene, an interleukin-12 gene and interferon genes.

The recombinant Ad-vectors and methods of the present invention may be used in the treatment or prevention of various respiratory pathogens. Such pathogens include, but are not limited to, influenza virus, severe acute respiratory syndrome-associated coronavirus (SARS-CoV), human rhinovirus (HRV), and respiratory syncytial virus (RSV).

In addition, the present methods comprehends the use of more than one therapeutic ligand, immunogen or antigen in the Ad-vectors and methods of the present invention, delivered either in separate recombinant vectors, or together in one recombinant vector so as to provide a multivalent vaccine or immunogenic composition that stimulates or modulates immunogenic response to one or more influenza strains and/or hybrids. Further, the present methods encompasses the use of a therapeutic ligand, immunogen or antigen from more than one pathogen in the vectors and methods of the present invention, delivered either in separate recombinant vectors, or together in one recombinant vector.

The methods of the invention may be appropriately applied to prevent diseases as prophylactic vaccination or treat diseases as therapeutic vaccination.

This disclosure relates to the use of a polynucleotide or TLR 3 agonist with Ad-vector vaccine as an adjuvant (Ad-vector vaccine adjuvant) for inducing an enhanced protective immune response in an animal in need thereof. In this instant, polynucleotides are molecular chains of nucleotides of ribonucleic acid (RNA). They may be of cellular or viral origin or they may be synthesized. Administering to an individual/subject/patient/animal the Ad-vector vaccine adjuvant appears to have at least four important functions. First, it has an immune stimulating effect (e.g. increasing the titer of neutralizing antibody to the antigen), second it increases mucosal immunity (e.g., increase in secretory IgA), third it increases interferon gamma producing cells (innate immunity), interferons are known to have an inhibitory effect upon viral infections and fourth it increases the safety of the Ad-vector vaccine as measured by a reduction in weight loss.

Interferons belong to the large class of glycoproteins known as cytokines They are natural proteins produced by the cells of the immune system in response to challenges by foreign agents such as viruses, parasites and tumor cells. Interferons are produced by a wide variety of cells in response to the presence of double-stranded RNA, a key indicator of viral infection. Interferons assist the immune response by inhibiting viral replication within host cells, activating natural killer cells and macrophages, increasing antigen presentation to lymphocytes, and inducing the resistance of host cells to viral infection. When the antigen is presented to matching T and B cells, those cells multiply, attack and degrade the infectious agent. Administering the Ad-vector vaccine adjuvant within 24 hours of the antigen (via Ad-vector vaccine) potentiates the immune response in addition to inducing the production of interferons.

In certain embodiments a natural dsRNA polynucleotide extracted from any number of known viral or bacterial agents is used. Such agents include influenza A virus, influenza B virus, Sendai virus, E. coli etc. The methods of extraction, amplification using polymerase chain reaction (PCR), and purification of the natural polynucleotide are known to those of skill in the art. A synthetic polynucleotide may also be used. Synthetic polynucleotides are double stranded nucleic acids selected from the group consisting of: polyinosinic acid and polycytidylic acid (poly-IC), polyadenylic acid and polyuridylic acid (poly-AU), polyinosinic acid analogue and polycytidylic acid, polyinosinic acid and polycytidylic acid analogue, polyinosinic acid analogue and polycytidylic acid analogue, polyadenylic acid analogue and polyuridylic acid, polyadenylic acid and polyuridylic acid analogue, and polyadenylic acid analogue and polyuridylic acid analogue.

The polynucleotide chain may be modified by substituting other bases into the chain at specified intervals, for example polyIC(12)U or polyIC(12)G, or by attaching additional compounds such as poly-L-lysine carboxymethylcellulose to the nucleotide chain. For example poly-IC may be stabilized by adding poly-L-lysine to form a new polynucleotide termed poly-ICLC.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

The Examples below are given so as to illustrate the practice of this invention. They are not intended to limit or define the entire scope of this invention.

### Example 1

Materials and Methods

Animals: Female 6 week-old BALB/c mice were obtained from Charles River Laboratories. The mice were quarantined for 72 hours before use and maintained on Teklad Rodent Diet (Harlan Teklad) and tap water at the Laboratory Animal Research Center of Utah State University.

Virus: Influenza A/Vietnam/1203/2004 (H5N1) was obtained from the Centers for Disease Control (Atlanta, Ga.). Viral propagation and assays were done in Madin-Darby canine kidney (MDCK) cells (American Type Culture Collection, Manassas, Va.). Parent virus was passaged once to prepare a challenge pool. The challenge pool was then titrated in MDCK cells before use. The cells were grown in MEM containing 5% fetal bovine serum (Hyclone, Logan, UT) and 0.18% sodium bicarbonate with no antibiotics in a 5% CO₂ incubator.

Vaccine: Ad5-VN1203/04.H5 (encoding the A/Vietnam/1203/04 H5 hemagglutinin gene) and the empty vector AdE, were prepared as described in (US Patent Publ. No., 2012/0276138, which reference is incorporated herein by reference in entirety). The virus titer for the AdVN.H5 was 7×10⁹ infection forming units (ifu)/ml (3.5×10⁸ ifu/0.05 ml) and AdE was 2.4×10⁹ ifu/ml (1.2×10⁸ ifu/0.05 ml). The vaccines were administered by the intranasal route in a 50 μl volume on a single occasion. Hiltonol® (synthetic dsRNA poly-ICLC, Oncovir, Inc.), used as vaccine adjuvant, preparation of poly-ICLC is described in (U.S. Pat. No. 7,439,349, incorporated by reference in entirety). The adjuvant was administered by the intranasal route in a 10 μl volume on a single occasion 30 minutes or 24 hours following administration of vaccine (see experimental design).

Experimental design: Animal numbers and study groups are described in Tables 1 and 2. Groups of mice were vaccinated on study day 0 by the intranasal route. The placebo groups received 50 μl physiological sterile saline (PSS) by the same route. Additional controls included mice vaccinated with the empty vector (AdE). For influenza virus challenge, mice were anesthetized by i.p. injection of ketamine/xylazine (50 mg/kg//5 mg/kg) prior to intranasal challenge with 50 μl of influenza A/Vietnam/1203/2004 (H5N1); approximately 5 plaque forming units (1× LD₉₀) of virus per mouse. All mice were administered virus challenge on study day 28. Following challenge all mice were observed for weight loss and mortality through day 21 post-challenge.

TABLE 1
Study Groups Used for Serological and Cytokine Analyses

| | | | Adjuvant | | | |
|---|---|---|---|---|---|---|
| No./ | Group | Vaccine Dose (IFU in 50 μl) | (Hiltonol ® | Adjuvant | | Observations/ |
| Cage | No. | (Ad5-VN1203/04.H5) | [μg] in 10 μl) | Administration | | Testing |
| 5 | 2 | Placebo (PSS) | — | — | | 2 mice sac on |
| 5 | 4 | Placebo (PSS) | 50 | 30 | minutes | D14, and 3 mice |
| | | | | | | sac on D28 |
| 13 | 6 | *AdE (1.2 × 10⁸) | — | — | | Day 14, five |
| 13 | 8 | *AdE (1.2 × 10⁸) | 50 | 30 | minutes | mice sacrificed |
| 13 | 10 | 1.2 × 10⁸ | — | — | | for lung lavage, |
| 13 | 12 | 1.2 × 10⁸ | 5 | 30 | minutes | spleen and |
| | | | | | | serum samples. |
| 13 | 14 | 1.2 × 10⁸ | 15 | 30 | minutes | Day 28, eight |
| 13 | 16 | 1.2 × 10⁸ | 50 | 30 | minutes | mice sacrificed |
| 13 | 18 | 1.2 × 10⁸ | 50 | 24 | hr | for lung lavage, |
| 13 | 20 | 1.2 × 10⁶ | 50 | 30 | minutes | spleen and |
| 13 | 22 | 1.2 × 10⁷ | 50 | 30 | minutes | serum samples. |
| 13 | 24 | 3.5 × 10⁸ | 50 | 30 | minutes | |

Empty vector in PSS.

Statistical analysis: Kaplan-Meier survival curves were generated and compared by the Log-rank (Mantel-Cox) test followed by pairwise comparison using the Gehan-Breslow-Wilcoxon test in Prism 5.0d (GraphPad Software Inc., La Jolla, Calif.). The mean body weights were analyzed by analysis of variance (ANOVA) followed by Tukey's multiple comparison test using Prism 5.0d. In addition, the results from serological assays (HAI, IgA, and adenovirus neutralization) and ELISpot assays were analyzed by analysis of variance (ANOVA) followed by Tukey's multiple comparison test using Prism 5.0d.

Hemagglutination inhibition (HAI) test: Serum samples were diluted in PBS in 96-well round-bottom microtiter plates (Fisher Scientific, Pittsburg, Pa.). Following dilution of serum, 8 HA units/well of influenza A/Vietnam/1203/2004 x Ann Arbor/6/60 hybrid virus (Vietnam H5 and N1 surface proteins and Ann Arbor core) plus chicken red blood cells (Lampire Biological Laboratories, Pipersville, Pa.) were added (50 μl of each per well), mixed briefly, and incubated for 60 min at room temperature. The HAI titers of serum samples are reported as the reciprocal of the highest serum dilution at which hemagglutination was completely inhibited.

TABLE 2
Expt. Study Groups Observed for Mortality and Changes in Body Weight

| | | | Vaccine Dose | Adjuvant | | | |
|---|---|---|---|---|---|---|---|
| No./ | Group | Infected | (IFU in 50 μl) | (Hiltonol ® | Adjuvant | | Observations/ |
| Cage | No. | Y or N | (Ad5-VN1203/04.H5) | [μg] in 10 μl) | Administration | | Testing |
| 10 | 1 | Yes | Placebo (PSS) | — | — | | Observed |
| 10 | 3 | Yes | Placebo (PSS) | 50 | 30 | minutes | for weight |
| 10 | 5 | Yes | *AdE (1.2 × 10⁸) | — | — | | loss and |
| 10 | 7 | Yes | *AdE (1.2 × 10⁸) | 50 | 30 | minutes | mortality |
| 10 | 9 | Yes | 1.2 × 10⁸ | — | — | | through day |
| 10 | 11 | Yes | 1.2 × 10⁸ | 5 | 30 | minutes | 21 post- |
| 10 | 13 | Yes | 1.2 × 10⁸ | 15 | 30 | minutes | challenge |
| 10 | 15 | Yes | 1.2 × 10⁸ | 50 | 30 | minutes | |
| 10 | 17 | Yes | 1.2 × 10⁸ | 50 | 24 | hr | |
| 10 | 19 | Yes | 1.2 × 10⁶ | 50 | 30 | minutes | |
| 10 | 21 | Yes | 1.2 × 10⁷ | 50 | 30 | minutes | |
| 10 | 23 | Yes | 3.5 × 10⁸ | 50 | 30 | minutes | |

| 10 | 25 | Yes | Ribavirin (75 mg/kg) | bid x 5 days, 12 hours apart, beg | |
|---|---|---|---|---|---|
| | | | | 4 hours post-challenge | |
| 10 | 27** | Yes | 3.5 × 10⁸ | 50 | 3 days before |
| | | | | | challenge |

| | | | |
|---|---|---|---|
| 5 | 26 | No | Normal controls observed for weight gain |

*Empty vector in PSS.

IgA ELISA: Total IgA levels in lung lavage samples from mice were determined by use of the mouse IgA enzyme immunoassay (EIA) kit (Bethyl Laboratories, Montgomery, Tex.) according to the manufacturer's instructions. Briefly, goat anti-mouse IgA bound to microtiter plates (Nunc MaxiSorp C; Fisher Scientific, Pittsburg, Pa.) was used to capture antibody from lavage fluid samples for 1 h at room temperature, after which goat anti-mouse IgA conjugated to horseradish peroxidase was used to detect bound antibody. Antibody concentrations were read off a standard curve generated by using pooled mouse sera calibrated for IgA antibody (Bethyl Laboratories).

ELISpot Assay for IFN-γ and IL-4: ELISpot kits for mouse IFN-γ and IL-4 (R&D Systems, Minneapolis, Minn.) were used according to the manufacturer's instructions. Briefly, lung lavage samples were harvested using 1.0 ml of sterile PBS containing 0.2 mM Pefabloc SC Plus (Hyclone, Logan, Utah). Cells from lung lavage samples were added to a 96-well cell culture plate at a concentration of 1.0×10⁵ cells/well suspended in 100 μl of RPMI-1640 with 2% FBS. Influenza A/California/04/2009 was diluted to approximately 1000 CCID₅₀/ml and 100 μl was added to each plate to achieve 100 CCID₅₀/well to stimulate production of cytokines The plates were incubated at 37° C. for approximately 24 hours. Following washing, 100 μl of Detection Antibody was added to each well and incubated overnight at 2-8° C. After washing, 100 μl of Streptavidin-AP was added to each well and incubated for 2 hours at room temperature. Following incubation, chromogen, 100 μl of BCIP/NBT, was added to each well and incubated for 1 hour at room temperature. After incubation, the chromogen solution was discarded and the plates washed with deionized water. The bottoms of the plates were air dried on paper towels and spots indicating cells actively producing cytokines were visually counted with a dissecting microscope.

Anti-Ad5 neutralizing antibody assay: HEK-293 cells were seeded in 96-well plates at 1×10⁴ cells per well in RPMI containing 10% FBS (Hyclone, Logan, Utah) 24 hours prior to use. On the next day, serial 2-fold dilutions of each serum sample were prepared in serum-free media starting at 1:10 dilution and ending at 1:1280. Each serum dilution was mixed 1:1 (0.1 ml) with serum-free media containing 1×10⁴ CCID₅₀/ml of wild type Adenovirus type 5 (American Type Culture Collection (ATCC), Manassas, Va.). After incubation at room temperature for 1 h, the serum-Ad5 mixture (0.2 ml) was transferred to a well containing 293 cells and incubated for 2 h. Following incubation, the serum-Ad5 mixture was removed and replaced with 0.1 ml of RPMI containing 0.5% FBS and gentamycin, then incubated for 3 days. Anti-Ad neutralizing antibodies were measured as cytopathic effect (CPE) inhibition. CPE was scored from duplicate samples by examining the 293 cell monolayers under a light microscope on day 3 post-infection.

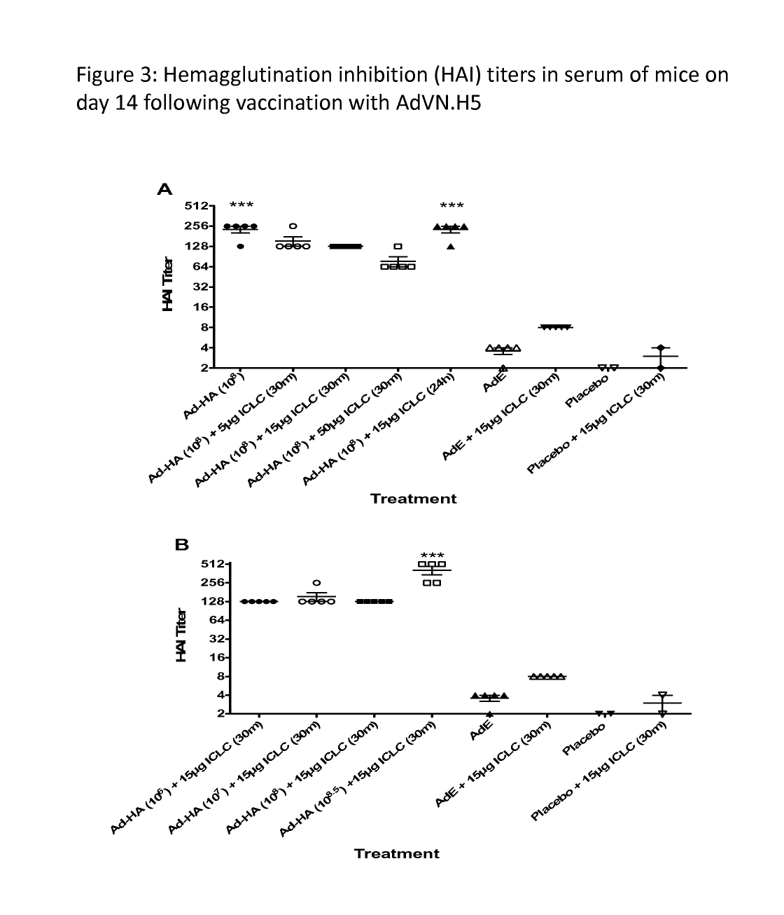

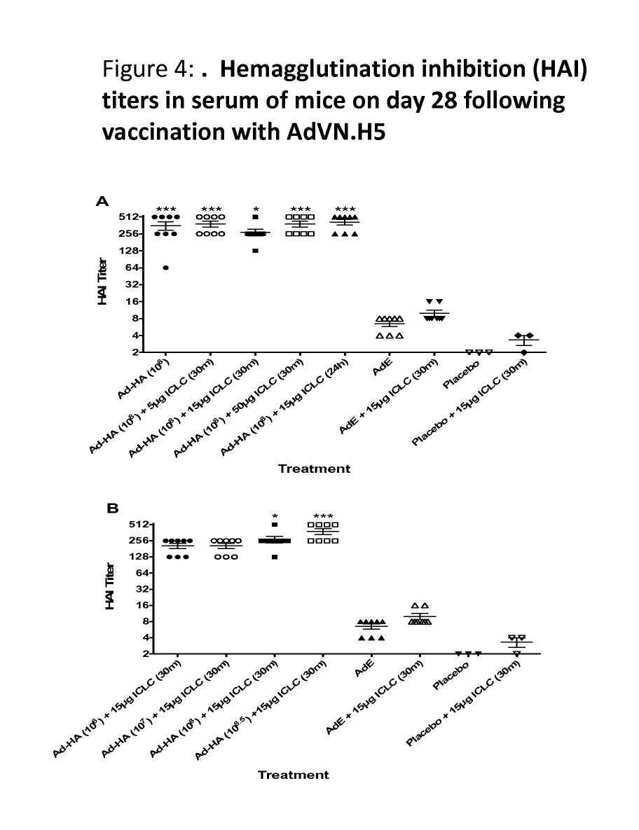

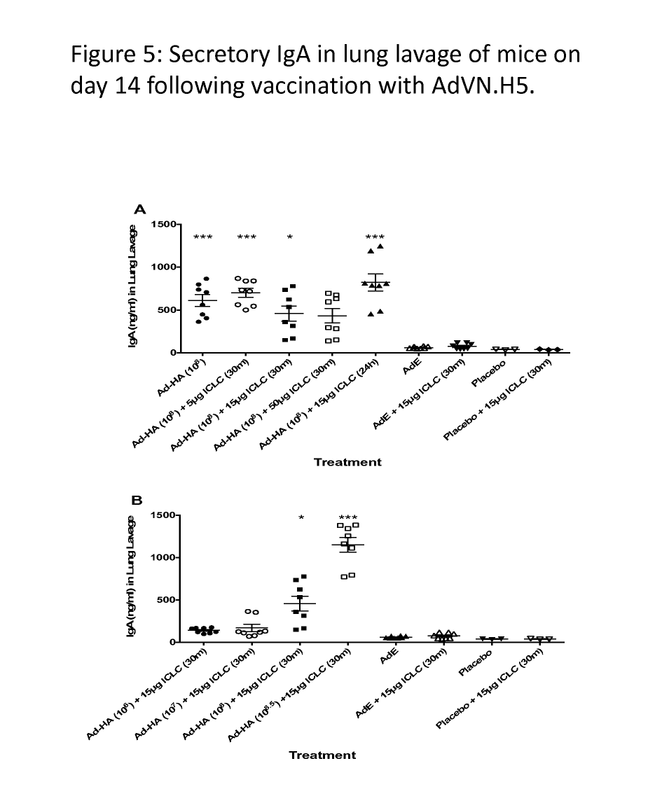

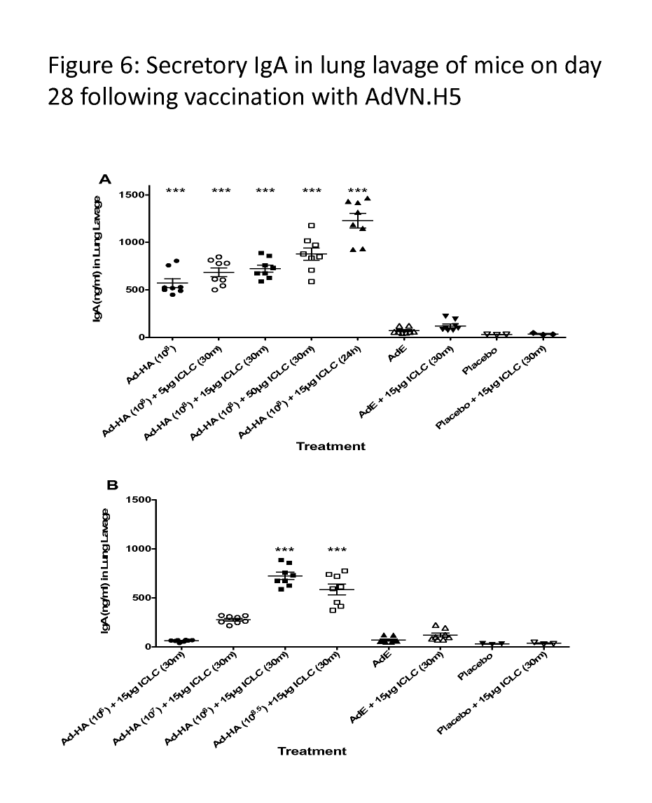

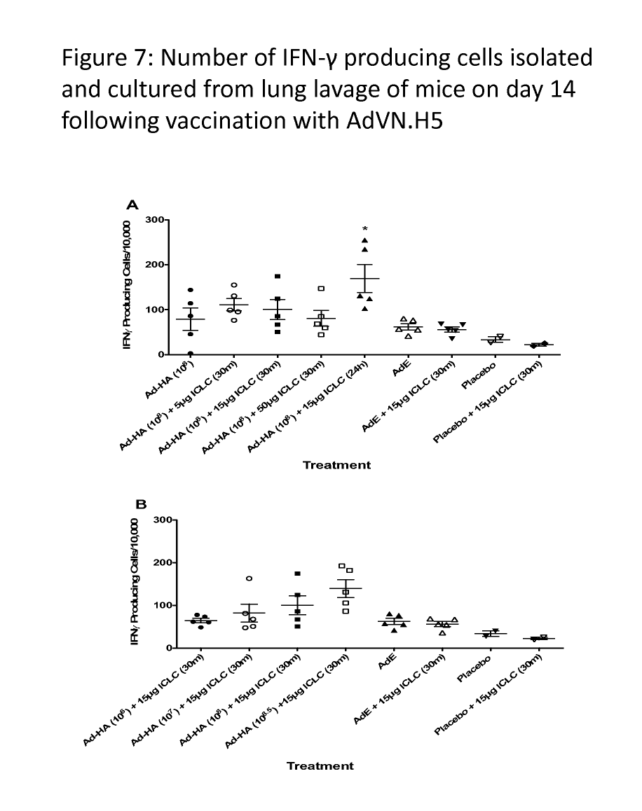

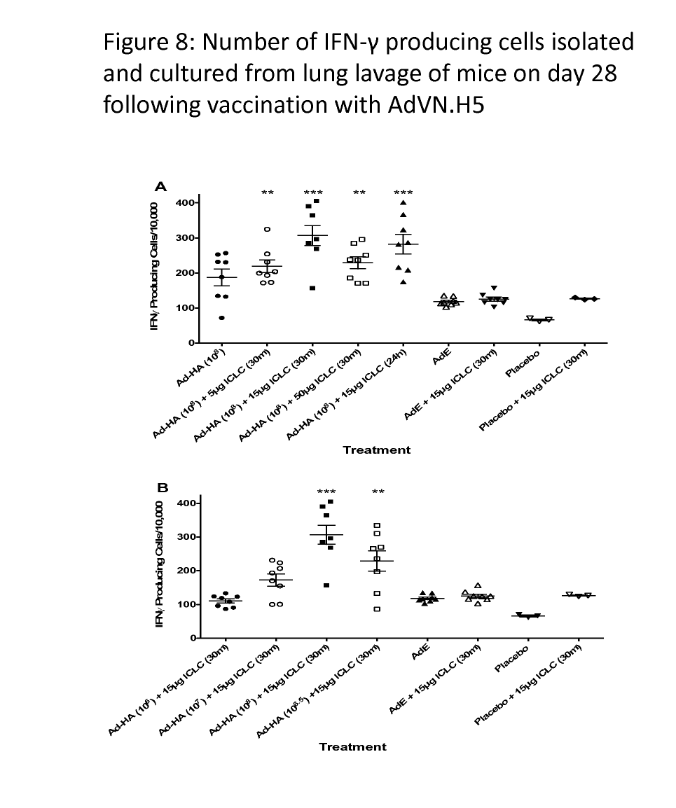

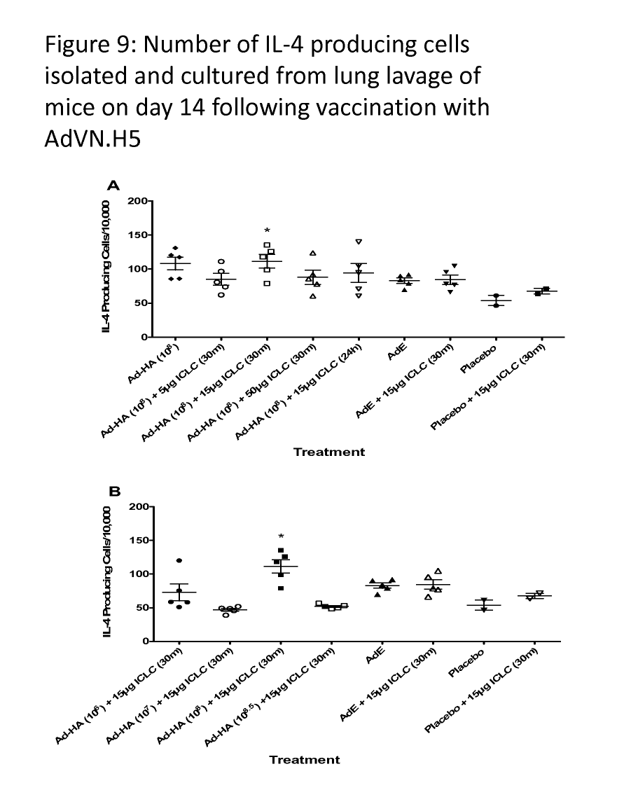

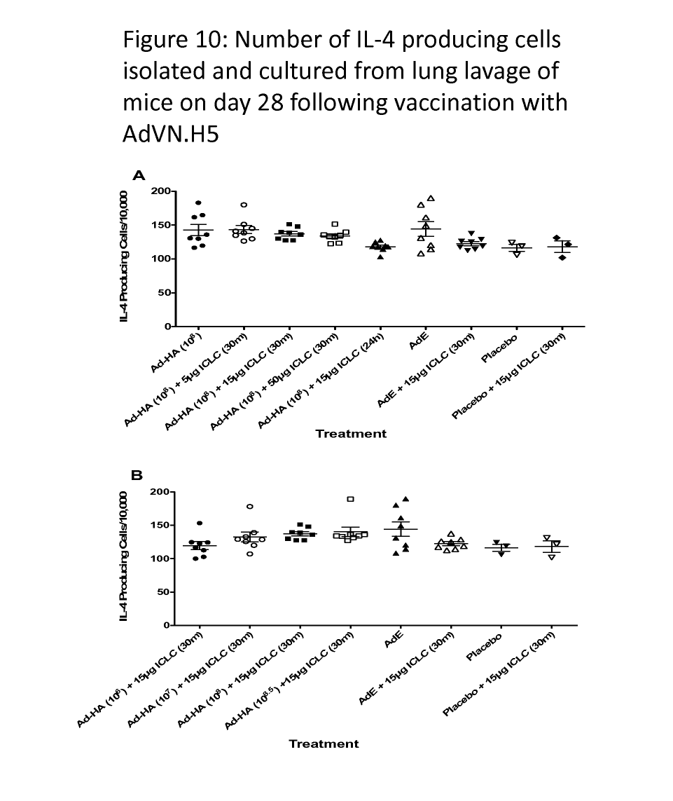

Evaluation of the immune response following vaccination included measurement of serum antibody levels by hemagglutination inhibition assay and secretory IgA (sIgA) levels in lung lavage. See FIGS. 3-6. Cellular immunity was evaluated by quantitation of cells, in lung lavage, releasing IFN-γ and IL-4 by ELISpot assay. See FIGS. 7-10

This study describes the use of a synthetic dsRNA poly-ICLC (Hiltonol®) as adjuvant to increase the immunogenicity of an Ad5-vectored influenza virus HA vaccine (Ad5-VN1203/04.H5) against challenge infection with highly pathogenic A/Vietnam/1203/04 (H5N1) avian influenza virus in mice. In a comparison of AdVN.H5 vaccines administered 30 min or 24 hours prior to administration of different doses of poly-ICLC, all treatment groups receiving the 10⁸ dose of Ad-VN.H5 provided 100% protection from challenge infection, regardless of the concentration of poly-ICLC. In addition, all four doses of AdVN.H5 (1.2×10⁶, 1.2×10⁷, 1.2×10⁸, or 3.5×10⁸ ifu/50 μl) vaccine administered 30 min prior to administration of 15 μg poly-ICLC provided 100% protection from challenge infection. The AdE also showed significant protection, although some mortality was observed. The protection afforded by the empty AdE vector was surprising, and suggests more than one mechanism of action for this specific Ad5 vector. All treatment groups receiving the 10⁸ dose of Ad-VN.H5 protected mice from significant weight loss, regardless of the concentration of poly-ICLC. However, the 5 μg dose of poly-ICLC showed the best protection. Upon comparing four doses of AdVN.H5 vaccines, the 10⁶ dose of AdVN.H5 combined with the 15 μg dose of poly-ICLC showed the best protection from weight loss. Therefore, the survival and weight loss data indicate that the 10⁸ dose of AdVN.H5 is protective regardless of the concentration of poly-ICLC. However, even lower doses of vaccine may be administered more safely and equally protective if combined with adjuvant.

Evaluation of the immune response following vaccination included measurement of serum antibody levels by hemagglutination inhibition assay and secretory IgA (sIgA) levels in lung lavage. Cellular immunity was evaluated by quantitation of cells, in lung lavage, releasing IFN-γ and IL-4 by ELISpot assay. Adenovirus-specific immunity was evaluated by adenovirus neutralization using serum from vaccinated mice.

A summary of immunological responses on days 14 and 28 post-vaccination follows:

All groups receiving the 10⁸ doses of AdVN.H5 induced significant levels of sIgA in lung lavage samples on day 14 post-vaccination. However, the 15 ug dose of poly-ICLC resulted in a higher sIgA titer than the 10⁸ dose of AdVN.H5 alone. On day 28 post vaccination, a 10⁸ dose of AdVN.H5 with 15 ug of polyICLC administered either 30 min or 24 hrs after the vector resulted in higher sIgA titers compared to a 10⁸ dose of AdVN.H5 alone.

On day 14 post-vaccination, only groups receiving the 10⁸ dose of AdVN.H5 combined with the 15 μg dose of poly-ICLC induced a significant increase in the number of IFN-γ producing cells isolated and cultured from lung lavage. However, by day 28 all groups receiving the 10⁸ dose of AdVN.H5 showed significant levels of IFN-γ producing cells. Thus, the inclusion of polyICLC increased the rate of immune response.

On day 14, only the treatment group receiving the 10⁸ dose of AdVN.H5 combined with the 15 μg dose of poly-ICLC showed a significant increase in the number of IL-4 producing cells. On day 28 post-vaccination, all treatment groups showed an increase in the number of IL-4 producing cells. Thus, the inclusion of polyICLC increased the rate of immune response.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method for increasing immunogenicity of an adenoviral vector (Ad-vector) vaccine in an animal, wherein the method comprises:
administering the Ad-vector in a non-invasive mode to the animal, wherein the vaccine comprises and expresses a gene of interest; and,
administering an Ad-vector vaccine adjuvant in a non-invasive mode to the animal within 24 hours of administering the Ad-vector vaccine, wherein the Ad-vector vaccine adjuvant is poly-ICLC or a TLR3 agonist,
wherein administration of the poly-ICLC or a TLR3 agonist increases the immunogenicity of the Ad-vector vaccine as compared to the Ad-vector vaccine administered without the poly-ICLC or a TLR3 agonist.

2. The method of claim 1, wherein the Ad-vector vaccine adjuvant is poly-ICLC.

3. The method of claim 1, wherein the increase in immunogenicity is measured by an increase in neutralizing antibody to the antigen as compared to the Ad-vector vaccine administered without the poly-ICLC or a TLR3 agonist.

4. The method of claim 1, wherein the increase in immunogenicity of the Ad-vector vaccine provides at least 90% protection against challenge from infection of the antigen.

5. The method of claim 1, wherein the increase in immunogenicity of the Ad-vector vaccine provides up to 100% protection against challenge from infection of the antigen wherein about at least 10⁶ ifu of the Ad-vector were administered to the animal.

6. The method of claim 1, wherein the increase in immunogenicity of the Ad-vector vaccine provides an antigen sparing effect.

7. The method of claim 1, wherein the non-invasive mode comprises skin administration, mucosal administration or intranasal administration.

8. The method of claim 1, wherein the animal is a human.

9. The method of claim 1, wherein the animal is a livestock animal.

10. The method of claim 1, wherein the livestock animal is a chicken, turkey, duck, or pig.

11. The method of claim 1, wherein the Ad-vector expresses a gene which encodes an antigen selected from the group consisting of influenza hemagglutinin, influenza nuclear protein, influenza neuraminidase, influenza M2, influenza M1, tetanus toxin C-fragment, anthrax protective antigen, anthrax lethal factor, rabies glycoprotein, HBV surface antigen, HIV gp 120, HW gp 160, malaria CSP, malaria SSP, malaria MSP, malaria pfg, mycobacterium tuberculosis HSP or a mutant thereof.

12. The method of claim 1, wherein the Ad-vector is E1/E3 defective adenovirus serotype 5 (Ad5).

13. The method of claim 1, wherein about 5ug to about 5 mg of poly-ICLC were administered to the animal.

14. The method of claim 1, wherein about 1 to about 2 mg of poly-ICLC were administered to the animal.

15. The method of claim 5, wherein about 1 to about 2 mg of poly-ICLC were administered to the animal.

16. The method of claim 1, wherein the TLR3 agonist is polyIC(12)U, polyIC(12)G or polyAU.

17. A non-invasive method for inducing a protective immune response in an animal in need thereof, wherein the method comprises:
administering the adenoviral vector (Ad-vector) in a non-invasive mode to the animal, wherein the vaccine comprises and expresses an antigen of interest; and,
administering an Ad-vector vaccine adjuvant in a non-invasive mode to the animal within 24 hours of administering the Ad-vector vaccine, wherein the Ad-vector vaccine adjuvant is poly-ICLC or a TLR3 agonist,
wherein induction of the immune response provides protection against challenge from infection of the antigen.

18. A method for increasing immune response rate to an adenoviral vector (Ad-vector) vaccine in an animal, wherein the method comprises:
administering the Ad-vector vaccine in a non-invasive mode to the animal, wherein the vaccine comprises and expresses an antigen of interest; and,
administering an Ad-vector vaccine adjuvant in a non-invasive mode to the animal within 24 hours of administering the Ad-vector vaccine, wherein the Ad-vector vaccine adjuvant is poly-ICLC or a TLR3 agonist,
wherein administration of the poly-ICLC or a TLR3 agonist increases the immune response rate to the Ad-vector vaccine as compared to an Ad-vectored vaccine administered without the poly-ICLC or a TLR3 agonist.
